(19) United States
(12) Patent Application Publication
(10) Pub. No.: US 2016/0058823 A1
(43) Pub. Date: Mar. 3, 2016

(54) COMPOSITION AND USE OF A DIETARY SUPPLEMENT

(71) Applicant: NEW MATRIX, BESLOTEN VENNOOTSCHAP MET BEPERKTE AANSPRAKELIJKHEID, Hasselt (BE)

(72) Inventor: Maria G. Baudry Jacquet, Hasselt (BE)

(21) Appl. No.: 14/782,046

(22) PCT Filed: Mar. 17, 2014
(86) PCT No.: PCT/BE2014/000012

§ 371 Date: Oct. 2, 2015

(30) Foreign Application Priority Data
Apr. 4, 2013 (DE) 2013/0237

(51) Int. Cl. A61K 38/01; A23L 1/305; A61K 31/185; A23L 1/30; A61K 36/258; A61K 36/42

(52) U.S. Cl. CPC A61K 38/018; A61K 36/258; A61K 36/42; A61K 31/185; A23L 1/3002; A23L 1/3053

(57) ABSTRACT

Composition of a dietary supplement, characterised in that it comprises a mixture of at least a-casein tryptic hydrolysate, Siberian or Asian Ginseng root powder, and lyophilised melon extract.

The present invention relates to a composition of a dietary supplement and the use and a dosage method thereof.

More specifically, the invention is intended to combat the symptoms of a professional exhaustion syndrome, also called “burnout” syndrome.

It is known that continually making extraordinary efforts on a professional level can lead to symptoms of physical and mental exhaustion, that can be summarised by the name “burnout” syndrome.

This burnout syndrome can be defined as a condition that is characterised by a set of signs, symptoms and behavioural changes in the professional environment. In general morphological, functional or biochemical changes are observed in people who suffer from it. As this condition is the result of a permanent and continuous exposure to professional stress, this burnout syndrome is placed in the category of psychosocial occupational hasards.

In order to combat these burnout symptoms, people sometimes resort to dietary supplements such as taking vitamins or minerals, but generally without obtaining a quick and adequate response.

The purpose of the present invention is to provide a solution to the aforementioned and other disadvantages by providing a dietary supplement that enables the symptoms of burnout to be fought.

The following table shows the active ingredients of the dietary supplement, shown as a quantity in mg/coated tablet, whereby a full coated tablet or pill weighs 640 mg and the normal administered dose is 2 coated tablets per day.

TABLE I
Active ingredients in the dietary supplement.

| Scientific | | Quantity | |
|---|---|---|---|
| name | Trivial name | mg/pill | Function |
| α-casein tryptic | Casozepine | 75 mg | Active |
| hydrolysate | | | ingredient |
| Eleutherococccus | Siberian or Asian | 50 mg | Active |
| senticosus L. | Ginseng | | ingredient |
| or Panax Ginseng | Root Powder | | |
| 2-aminoethane- | Taurine | 50 mg | Active |
| sulfonic acid | | | ingredient |
| Cucumis melo | Extramel | 5 mg | Active |
| L. | Lyophilised | | ingredient |
| | Melon Extract | | |

The following table shows the other ingredients, in a 640 mg coated tablet, together with the function that they have in the dietary supplement.

TABLE II
non-active ingredients in the dietary supplement.

| Scientific | | Quantity | | |
|---|---|---|---|---|
| name | Trivial name | mg/pill | | Function |
| Microcrystalline | Cellulose | 400 | mg | Thickening |
| cellulose | | | | agent |
| Mg-stearate | Magnesium | 20 | mg | Anticlotting agent |
| | stearate | | | |
| SiO2 | Silicon dioxide | 20 | mg | Anticlotting agent |
| Vegetable | Dehydrogenated | 10 | mg | Glazing agent |
| oil | vegetable oil | | | |
| Hypromellose | Hypromellose | 3 | mg | Filler |
| Polydextrose | Polydextrose | 3 | mg | Glazing agent |
| TiO2 | Titanium dioxide | 4.15 | mg | Colouring agent |
| Propylene | Propylene glycol | 0.35 | mg | Glazing agent |
| glycol | E1520 | | | |
| Riboflavin- | Riboflavin-5- | 0.05 | mg | Colouring agent |
| 5-phosphate | phosphate E101 | | | |
| Carmine | Carmine E120 | 0.03 | mg | Colouring agent |

The present invention concerns a dietary supplement, consisting of a mixture of at least α-casein tryptic hydrolysate, Siberian or Asian Ginseng root powder and lyophilised melon extract.

Preferably taurine is also added to the composition as a fourth active ingredient.

The present invention also concerns the use of a dietary supplement, with at least oc-casein tryptic hydrolysate, Siberian or Asian Ginseng root powder and lyophilised melon extract for fighting burnout syndrome.

In order to evaluate the efficacy of this dietary supplement against burnout and to test the tolerance to it, a clinical trial was organised in the department of clinical pharmacology of the University of Bordeaux over a period of one year.

It was a randomised, double-blind test on two parallel groups, in which 87 volunteers participated. This means that
half of the volunteers were given the dietary supplement according to the invention. We refer to this product hereinafter as the “verum”.
the other half of the volunteers received a placebo product, i.e. a product without pharmacological effect. We refer to this product hereinafter as the “placebo”.
the volunteer did not know whether he was taking the “placebo” or the “verum” product.
the trial physician did not know whether he was giving a placebo or a verum product, which is why the test is called a double-blind test.

In this test the “verum” product was administered orally every morning in the form of two 640 mg coated tablets with the above composition, and this every morning for 84 days.

The products were distributed according to a predetermined list drawn up by the centre of statistical analysis, which enabled a homogeneous distribution of the “placebo” and “verum”; this is the ‘randomisation’.

This list, which indicates which volunteer numbers had to be given a “placebo” and which numbers a “verum”, could only be made known after the definitive results of the test had been given to the analysis centre, so that the double-blind nature was safeguarded.

This methodology is used most frequently for the clinical tests of dietary supplements. For the tests concerning this range of products, this is almost a requirement to obtain the approval of the ministerial authorities, at least in France.

The methodology comprises the following steps:
87 volunteers presenting clinical signs of burnout were included in the test (55 women and 32 men);
they all have a profession in which they come into direct contact with patients or pupils or customers. This requirement of direct contact with patients, pupils or customers is to do with the fact that one of the international validated scales used in this test has been specifically designed for this type of profession and is not suitable for professions that do not have direct contact with these groups of patients, pupils or customers, i.e. the MBI-HSS scale;
each volunteer was seen and examined up to three times: D0, D42 and D84, i.e. on day 0, after 42 days and after 84 days of treatment. At each examination measurements were taken of:
the BMS-10 burnout scale: the abridged version of Maslach and Pines. Ten items are scored from 1 to 7 according to their frequency. These scores are determined by adding up the value of each item and dividing by ten. They thus vary from 1 to 7. In order to be included in this test programme, the score must be at least 4, which confirms the existence of burnout.
the Maslach burnout inventory scale (MBI-HSS): this comprises 22 items that are scored from 0 to 6 according to their frequency, and which are divided into three groups:
burnout (9 items): varies between 0 and 54,
depersonalisation (5 items): between 0 and 30,
the completion of tasks (8 items): between 0 and 48.
the Beck depression scale: this comprises 21 items that are scored between 0 and 3 according to their intensity. The score thus varies between 0 and 63. It enables the importance of the depression to be quantified that results from the burnout and is associated with it.

The results of these three scales were statistically processed to analyse the following:
the development of each item within each group and this for the administration of the verum and the placebo;
the comparison of the values of the scores between the verum and placebo groups;
the table mentioned below only gives a summary of the statistical analysis, in which only the average values and the conclusions of statistical significance are reported.

The differences between the scores on day 0 and day 84 were calculated statistically for the individual scores of each volunteer and not for the average scores. The differences between day 0 and 48 are thus not differences of averages but averages of the differences.

To this end the score on day 0 was subtracted from the scores after 84 days for each volunteer, after which the average of all these differences was calculated.

In order to present the still unpublished results of this double-blind study, they are explained in the accompanying diagram, as an example without any limiting nature:

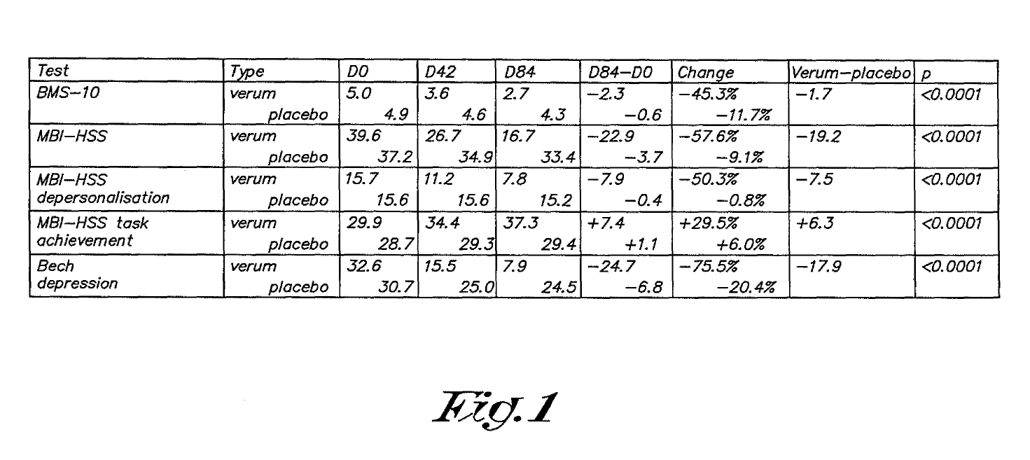

FIG. 1 shows in tabular form the measured results of the effects of the composition according to the invention on burnout syndrome.

FIG. 1 shows the measured data in tabular form and this for five test methods that are identified in the first column, which each yield a score relating to the burnout syndrome. The second column indicates whether the test concerned was taken after the administration of the dietary supplement according to the invention of the “verum” type, or after the administration of a “placebo” type without active ingredients. The third to fifth columns show the measured scores at the start of the test period (day 0), halfway through the test period (day 42) and at the end of the test period (day 84). The sixth column shows the difference of the measured value at the end of the test to the starting value of the test on day 0. The seventh column shows this difference as a percentage of the starting value.

The eighth column shows the difference for the “verum” score, from which the difference for the “placebo” score was subtracted, i.e. the net difference attributable to the dietary supplement. The ninth column shows the statistical significance of this net difference, which shows that the probability that the measured result is coincidental for all test methods is less than 1 in 10,000. This means that the measured effect of the dietary supplement on burnout syndrome can be called highly significant.

Also for other criteria than the said five test methods, the established effect of the composition according to the invention is statistically highly significant (p<0.0001), such as for the following criteria:
the quality of the professional life;
the quality of family life;
the quality of the night's sleep;
the energy level of the patient.

Moreover, it was found that the tolerance to the dietary supplement turned out to be excellent.

The coated tablets can be administered daily over an extended period and the quantity of active ingredient per coated tablet can also vary between:
35 mg and 225 mg for the α-casein tryptic hydrolysate
25 mg and 150 mg for the Siberian or Asian Ginseng root powder;
2.5 mg and 15 mg for the lyophilised melon extract;

and optionally between:

25 mg and 150 mg for 2-aminosulfonic acid.

The present invention also concerns a dosing method for a dietary supplement with a composition according to the invention, whereby the dietary supplement is administered orally in the form of one or more coated tablets over a period of one or more weeks or months.

The present invention is by no means limited to the composition described as an example, but a dietary supplement according to the invention with at least the three active ingredients described in the first claim can be used in all kinds of compositions and doses, without departing from the scope of the invention.

1. Composition of a dietary supplement, characterised in that it comprises a mixture of at least α-casein tryptic hydrolysate, Siberian or Asian Ginseng root powder, and lyophilised melon extract.

2. Composition according to claim 1, characterised in that 2-aminoethanesulfonic acid is added to the mixture.

3. Composition according to claim 1, characterised in that the quantity of active ingredient per coated tablet:
is between 35 mg and 225 mg and preferably 75 mg for the a-casein tryptic hydrolysate;
between 25 mg and 150 mg and preferably 50 mg for the Siberian or Asian Ginseng root powder;
between 2.5 mg and 15 mg and preferably 5 mg for the het lyophilised melon extract;

4. Composition according to claim 2, characterised in that the quantity of 2-aminoethanesulfonic acid per coated tablet:
is between 25 mg and 150 mg and preferably 50 mg.

5. Use of a dietary supplement, with at least a-casein tryptic hydrolysate, Siberian or Asian Ginseng root powder, and lyophilised melon extract to combat burnout syndrome.

6. Dosing method for a dietary supplement according to claim 1, characterised in that the dietary supplement is administered orally in the form of one or more coated tablets per day over a period of one or more weeks or months.
